(19) United States
(12) Patent Application Publication
(10) Pub. No.: US 2024/0293678 A1
(43) Pub. Date: Sep. 5, 2024

(54) METHOD AND APPARATUS FOR USING AGGREGATE PATIENT DATA IN NEUROSTIMULATION DEVICE SETUP AND/OR PROGRAMMING

(71) Applicant: Boston Scientific Neuromodulation Corporation, Valencia, CA (US)

(72) Inventor: Lisa Denise Moore, Glendale, CA (US)

(21) Appl. No.: 18/584,545

(22) Filed: Feb. 22, 2024

Related U.S. Application Data
(60) Provisional application No. 63/449,368, filed on Mar. 2, 2023.

(51) Int. Cl. A61N 1/36; A61N 1/372

(52) U.S. Cl. CPC A61N 1/36139; A61N 1/36146; A61N 1/37247

(57) ABSTRACT

A neurostimulation system uses aggregate patient data for setting up and programming a stimulation device for a patient. In an example, the system may include a programming control circuit and a user interface. The programming control circuit may be configured to generate information for programming the stimulation device to control delivery of neurostimulation according to a stimulation configuration. The user interface may include a stimulation programming circuit configured to receive aggregate data collected from a patient population, to receive patient-specific data collected from the patient, to select relevant data from the received aggregate data according to selection criteria, to generate a target recommendation based on the selected relevant data and the patient-specific data, to determine a data presentation based on the selected relevant data and the patient-specific data, and to determine the stimulation configuration using the target recommendation.

### CLAIM OF PRIORITY

This application claims the benefit of U.S. Provisional Application No. 63/449,368 filed on Mar. 2, 2023, which is hereby incorporated by reference in its entirety.

### TECHNICAL FIELD

This document relates generally to neurostimulation and more particularly to a neurostimulation system that processes aggregate data collected from a patient population for use in setting up and/or programming a stimulation device for a patient.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Various types of data can be collected from the patient as well as a patient population and used in, for example, determining candidacy of the patient for neurostimulation, recommending initial values of the stimulation parameters to start a neurostimulation therapy for the patient, and adjusting the values of the stimulation parameters for titration of the neurostimulation therapy.

### SUMMARY

An Example (e.g., “Example 1”) of a system for delivering neurostimulation to a patient using a stimulation device configured to deliver the neurostimulation is provided. The system may include a programming control circuit and a user interface. The programming control circuit may be configured to generate information for programming the stimulation device to control the delivery of the neurostimulation according to a stimulation configuration including stimulation parameters defining one or more stimulation waveforms and one or more stimulation fields. The user interface may include a presentation device, a user input device, and a stimulation programming circuit coupled to the presentation device and the user input device and configured to receive aggregate data collected from a patient population, to receive patient-specific data collected from the patient, and to determine the stimulation configuration. The stimulation programming circuit may include data selection circuitry configured to select relevant data from the received aggregate data according to selection criteria, recommendation circuitry configured to generate a target recommendation based on the selected relevant data and the patient-specific data, presentation control circuitry configured to determine a data presentation using the presentation device based on the selected relevant data and the patient-specific data, and stimulation configuration circuitry configured to determine the stimulation configuration using the target recommendation.

In Example 2, the subject matter of Example 1 may optionally be configured such that the data selection circuitry is further configured to determine the selection criteria automatically using the received patient-specific data and information required for generating the target recommendation.

In Example 3, the subject matter of Example 2 may optionally be configured such that the data selection circuitry is configured to determine the selection criteria for selecting the relevant data based on profiles including collective patient profiles and a personal profile of the patient. The collective patient profiles are each extracted from the aggregate data for a patient of the patient population. The personal profile is extracted from the patient-specific information. Each profile of the collective patient profiles and the personal profile includes at least one of diagnostic information or therapeutic information.

In Example 4, the subject matter of Example 3 may optionally be configured such that the data selection circuitry is configured to determine the selection criteria for selecting the relevant data from patients having similar profiles.

In Example 5, the subject matter of any one or any combination of Examples 3 and 4 may optionally be configured such that the data selection circuitry is further configured to determine the selection criteria for selecting the relevant data based on the profiles and specified parameter ranges.

In Example 6, the subject matter of any one or any combination of Examples 3 to 5 may optionally be configured such that the data selection circuitry is further configured to determine the selection criteria for selecting the relevant data further based on a number of qualified patients of the patient population.

In Example 7, the subject matter of any one or any combination of Examples 1 to 6 may optionally be configured such that the recommendation circuitry is configured to generate an input recommendation of the target recommendation, and the stimulation configuration circuitry is configured to execute an algorithm configured to determine an output recommendation automatically based on the input recommendation, to receive a user command, and to determine the stimulation configuration using the output recommendation and the user command.

In Example 8, the subject matter of any one or any combination of Examples 1 to 7 may optionally be configured such that the recommendation circuitry is configured to automatically generate target recommendation based on the selected relevant data and the patient-specific data.

In Example 9, the subject matter of any one or any combination of Examples 1 to 8 may optionally be configured such that the presentation circuitry is configured to present visual representation of at least one of portions of the selected relevant data or data derived from the selected relevant data using the presentation device.

In Example 10, the subject matter of any one or any combination of Examples 1 to 9 may optionally be configured such that the presentation circuitry is configured to present visual representation showing overlapping between the target recommendation and corresponding current state of the system.

In Example 11, the subject matter of any one or any combination of Examples 1 to 10 may optionally be configured such that the presentation circuitry is configured to present a measure of likelihood of an intended outcome of the delivery of the neurostimulation determined based on a comparison of the target recommendation to the corresponding current state of the system.

In Example 12, the subject matter of any one or any combination of Examples 1 to 11 may optionally be configured such that the stimulation configuration circuitry is configured to receive a user command using the user input device and to generate the stimulation configuration based on the target recommendation and the user command.

In Example 13, the subject matter of any one or any combination of Examples 1 to 12 may optionally be configured such that the presentation control circuit is configured to determine the data presentation using portions of the selected relevant data for guiding lead placement during implantation of the stimulation device. The data presentation includes stimulation field models associated with lead positions collected from patients of the patient population having implanted stimulation devices. The stimulation field models (SFMs) each represent a volume of tissue activated by the delivery of the neurostimulation.

In Example 14, the subject matter of any one or any combination of Examples 1 to 13 may optionally be configured such that the presentation control circuit is configured to determine the data presentation using portions of the selected relevant data for guiding an identification of a stimulation target being an area or a volume of tissue to which the neurostimulation is to be delivered. The data presentation includes prior stimulation targets associated with lead positions collected from patients of the patient population having implanted stimulation devices.

In Example 15, the subject matter of any one or any combination of Examples 1 to 14 may optionally be configured such that the presentation control circuit is configured to determine the data presentation using portions of the selected relevant data. The data presentation includes setup features determined using the portions of the selected relevant data and used as input used for generating the target recommendation automatically.

An example (e.g., “Example 16”) of a method for delivering neurostimulation to a patient using a stimulation device configured to deliver the neurostimulation is also provided. The method may include: receiving aggregate data collected from a patient population, receiving patient-specific data collected from the patient, selecting relevant data from the received aggregate data according to selection criteria using a processor, generating a target recommendation based on the selected relevant data and the patient-specific data using the processor, determining a data presentation based on the selected relevant data and the patient-specific data using the processor, to be presented using a user interface, determining a stimulation configuration using the target recommendation using the processor, and programming the stimulation device to control the delivery of the neurostimulation according to the stimulation configuration. The stimulation configuration may include stimulation parameters defining one or more stimulation waveforms and one or more stimulation fields.

In Example 17, the subject matter of determining the data presentation as found in Example 16 may optionally include determining a visual representation of at least one of portions of the selected relevant data or data derived from the selected relevant data using the presentation device.

In Example 18, the subject matter of determining the data presentation as found in any one or any combination of Examples 16 and 17 may optionally include determining a visual representation showing overlapping between the target recommendation and corresponding current state of the system.

In Example 19, the subject matter of determining the data presentation as found in any one or any combination of Examples 16 to 18 may optionally include determining a visual representation including a measure of likelihood of an intended outcome of the delivery of the neurostimulation determined based on a comparison of the target recommendation to the corresponding current state of the system.

In Example 20, the subject matter of any one or any combination of Examples 16 to 19 may optionally further include determining the selection criteria for selecting the relevant data based on profiles including collective patient profiles and a personal profile of the patient. The collective patient profiles are each extracted from the aggregate data for a patient of the patient population. The personal profile is extracted from the patient-specific information. Each profile of the collective patient profiles and the personal profile includes at least one of diagnostic information or therapeutic information.

In Example 21, the subject matter of determining the selection criteria as found in Example 20 may optionally include determining the selection criteria for selecting the relevant data further based on at least one of specified parameter ranges or a number of qualified patients of the patient population.

In Example 22, the subject matter of determining the data presentation as found in any one or any combination of Examples 16 to 21 may optionally include determining the data presentation using portions of the selected relevant data for guiding lead placement during implantation of the stimulation device. The data presentation includes stimulation field models associated with lead positions collected from patients of the patient population having implanted stimulation devices. The stimulation field models (SFMs) each represent a volume of tissue activated by the delivery of the neurostimulation.

In Example 23, the subject matter of determining the data presentation as found in any one or any combination of Examples 16 to 22 may optionally include determining the data presentation using portions of the selected relevant data for guiding an identification of a stimulation target being an area or a volume of tissue to which the neurostimulation is to be delivered. The data presentation includes prior stimulation targets associated with lead positions collected from patients of the patient population having implanted stimulation devices.

In Example 24, the subject matter of determining the data presentation as found in any one or any combination of Examples 16 to 23 may optionally include determining the data presentation using portions of the selected relevant data. The data presentation includes setup features determined using the portions of the selected relevant data and used as input used for generating the target recommendation automatically.

An example (e.g., “Example 25”) of a non-transitory computer-readable storage medium including instructions is also provided. The instructions, which when executed by a system, cause the system to perform a method for delivering neurostimulation to a patient using a stimulation device configured to deliver the neurostimulation. The method may include: receiving aggregate data collected from a patient population, receiving patient-specific data collected from the patient, selecting relevant data from the received aggregate data according to selection criteria, generating a target recommendation based on the selected relevant data and the patient-specific data, determining a data presentation based on the selected relevant data and the patient-specific data using the processor, to be presented using a user interface, determining a stimulation configuration using the target recommendation, and generating information for programming the stimulation device to control the delivery of the neurostimulation according to the stimulation configuration. The stimulation configuration may include stimulation parameters defining one or more stimulation waveforms and one or more stimulation fields.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a stimulation programming circuit of a programming device of a neurostimulation system, such as the programming device of FIG. 3 or FIG. 8. The stimulation programming circuit can be configured for determining settings of a stimulation device for a patient using aggregate data collected from a patient population and patient-specific information collected from the patient.

FIG. 10 illustrates an embodiment a neurostimulation system communicatively coupled to various databases via a telecommunication system.

FIG. 11 illustrates an embodiment of a user interface of an external programming device of an implantable neurostimulation system, such as the external programming device of FIG. 10.

FIG. 12 illustrates an embodiment of portions of a display for guiding lead placement on a user interface of an implantable neurostimulation system, such as the user interface of FIG. 11.

FIG. 13 illustrates an embodiment of portions of a display for identifying stimulation targets on a user interface of an implantable neurostimulation system, such as the user interface of FIG. 11.

FIG. 14 illustrates an embodiment of portions of a display for recommending setup features on a user interface of an implantable neurostimulation system, such as the user interface of FIG. 11.

FIG. 15 illustrates an embodiment of a method for determining settings of a stimulation device for a patient using aggregate data collected from a patient population and patient-specific information collected from the patient.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that receives aggregate data collected from a patient population for use in programming a stimulation device to deliver neurostimulation to an individual patient. The system can select relevant data from the received aggregate data according to criteria specified for the individual patient, for example based on diagnostic information and therapeutic goals of that patient. The system can then present the selected relevant data and/or generate recommendations in a manner facilitating the process of programming the stimulation device for the individual patient. In various embodiments, the neuromodulation system can include an implantable device configured to deliver neurostimulation (also referred to as neuromodulation) therapies, such as deep brain stimulation (DBS), spinal cord stimulation (SCS), peripheral nerve stimulation (PNS), and vagus nerve stimulation (VNS), and one or more external devices configured to program or adjust the implantable device for its operations and monitor the performance of the implantable device.

In this document, unless noted otherwise, a “patient” includes a person receiving treatment delivered from, and/or being monitored and/or evaluated using, a neurostimulation system. A “user” includes a physician, other caregiver who examines, monitors, and/or treats the patient using the neurostimulation system, or other person who participates in the examination, monitoring, and/or treatment of the patient using the neurostimulation system (e.g., a technically trained representative, a field clinical engineer, a clinical researcher, or a field specialist from the manufacturer of the neurostimulation system).

Aggregate data useful for determining whether and how to treat a patient indicated for neurostimulation can be collected from other patients who have been treated using neurostimulation for similar indications. The aggregate data can include data directly collected from the patients. For example, a patient application can be installed on a patient's smartphone to present a questionnaire and receive answers and/or to receive signals sensed by one or more sensors worn by the patient. A remote controller can be provided to the patient for adjusting delivery of neurostimulation from an implantable stimulator placed in the patient. The remote controller can receive signals sensed by the implantable stimulator and record adjustments to the settings of the implantable neurostimulators made by the patient. The aggregate data can include data indirectly collected from the patients. For example, an external device (e.g., programmer) communicating with an implantable stimulator placed in a patient can receive signals sensed by the implantable stimulator, receive information tracking operations of the implantable stimulator, record settings of the implantable stimulator as programmed using the external device, and/or record information used by the external device for determining the settings of the implantable stimulator. The aggregate data can be collected through multiple users and/or user groups (e.g., clinics and/or specialty networks) of stimulation devices such as implantable stimulators.

The aggregate data can be used to assist programming settings of the implantable stimulator for each individual patient. In one example, the aggregate data can be used to generate recommendations for the settings for each individual patient based on settings known to be effective for other patients sharing common and/or similar pathological conditions and device characteristics. For SCS and DBS, computerized tools have been developed that read various default or recommended settings (e.g., stimulation target and/or stimulation parameters) and patient-specific information (i.e., information about the individual patient, such as the patient's symptoms) as inputs based on which the recommendations for the settings of an implantable stimulator can be generated, and the aggregate data can be used to set the various default or recommended settings. Then, the user can determine whether to accept the recommendations in programming the implantable stimulator. Using the aggregate data to determine the various default settings can serve as a good starting point in determining the settings of the implantable stimulator for the patient based on knowledge accumulated from the practices of treating numerous other patients. When determining or adjusting such default settings, the user may have to study a large volume of aggregate data to benefit from the accumulated knowledge.

The present subject matter processes the aggregate data collected from the patient population to extract information for use in determining settings of a stimulation device that delivers neurostimulation to a patient. In various embodiments, relevant data can be selected from the collected aggregate data based on purposes related to the patient's profile (e.g., including diagnostic data and settings of the stimulation device). The selected relevant data can include data collected from patients having similar profiles and can be, for example, presented to the user, used to generate information fed into a computerized tool that recommends settings of the stimulation device to the user, and/or used with patient-specific information in an automatic process of determining the settings of the stimulation device. In examples such as these, the present subject matter can be applied to increase utilization of tools generating recommendation for the settings of the stimulation device, increase confidence in the recommendations generated by the tools, and/or increase efficacy, safety, and patient satisfaction of the therapy delivered using the stimulation device.

The present subject matter can be applied at each stage of the therapy that using the stimulation device. For example, when the stimulation device is an implantable stimulator, the aggregate data can be used before, during, and after implantation of device. The pre-implant application can include using a mobile application installed in a smartphone for medication management, survey (questions for evaluating the patient), and receiving and processing signals from wearable sensors to collect patient-specific data for analysis with the relevant data selected from the aggregate data to determine whether the patient will likely benefit from using the implantable stimulator. The during-implant application can include using the relevant data selected from the aggregate data with the patient-specific data to generate recommendations for programming the implantable stimulator and/or positioning stimulation electrodes. The post-implant application can include using the relevant data selected from the aggregate data with the patient-specific data to generate recommendations for adjusting settings of the implantable stimulator as needed, with data acquired using the implantable stimulator since the implantation added to the patient-specific data and/or with the aggregate data updated (e.g., to include newly collected data).

Examples of applying the present subject matter include processing the collected aggregate data for:
A. guiding lead placement (electrode positioning), e.g., for DBS and SCS;
B. identifying stimulation targets, e.g., based on symptoms and patient anatomy, for using an anatomy-based programming tool that receives stimulation targets, lead position, and areas to avoid as input for recommending settings for an implantable stimulator; and
C. recommending setup features, e.g., default settings for the implantable stimulator based on symptoms, lead position, and search type, for using a programming tool that receives setup features as input to a search algorithm for exploring various settings of the implantable stimulator to recommend optimal settings for testing by delivering neurostimulation to the patient upon acceptance by the user.

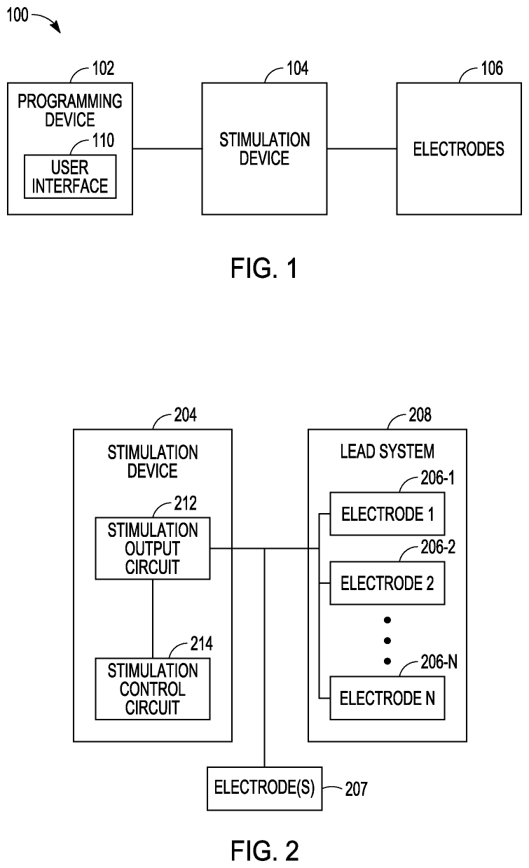

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact in this document), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

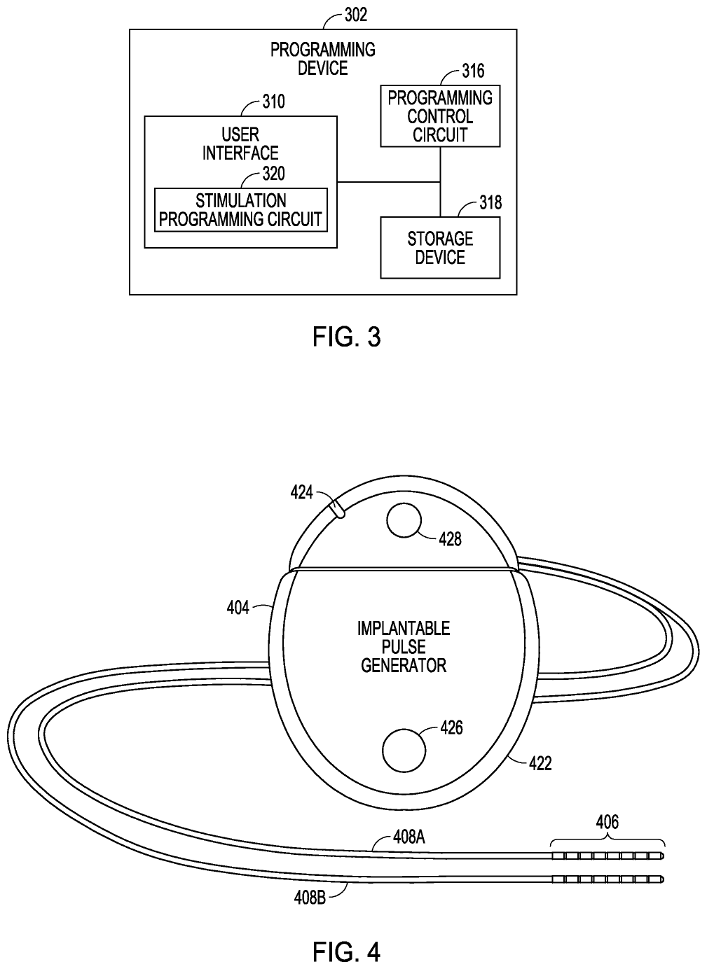

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions supporting the programming of stimulation devices, such as stimulation device 104 including its various embodiments as discussed in this document, including various uses of aggregate data collected from a patient population as discussed in this document.

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulation programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes (also referred to as contacts) 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

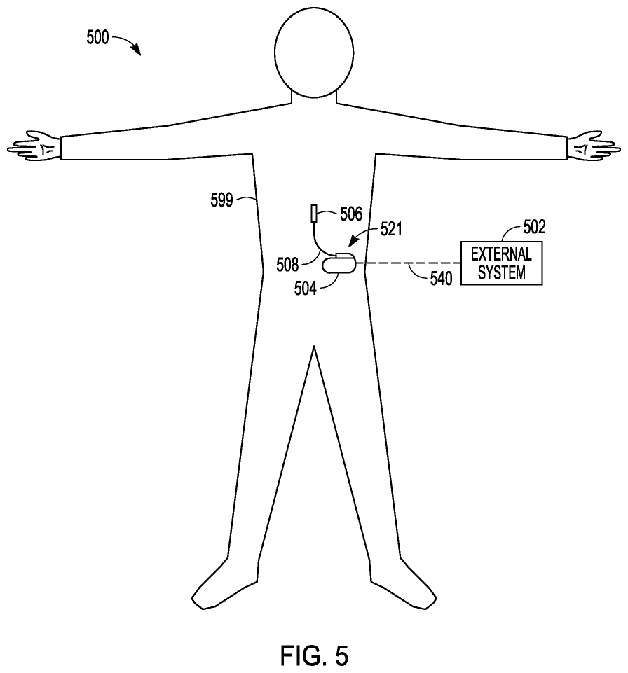

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes (also referred to as contacts) 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

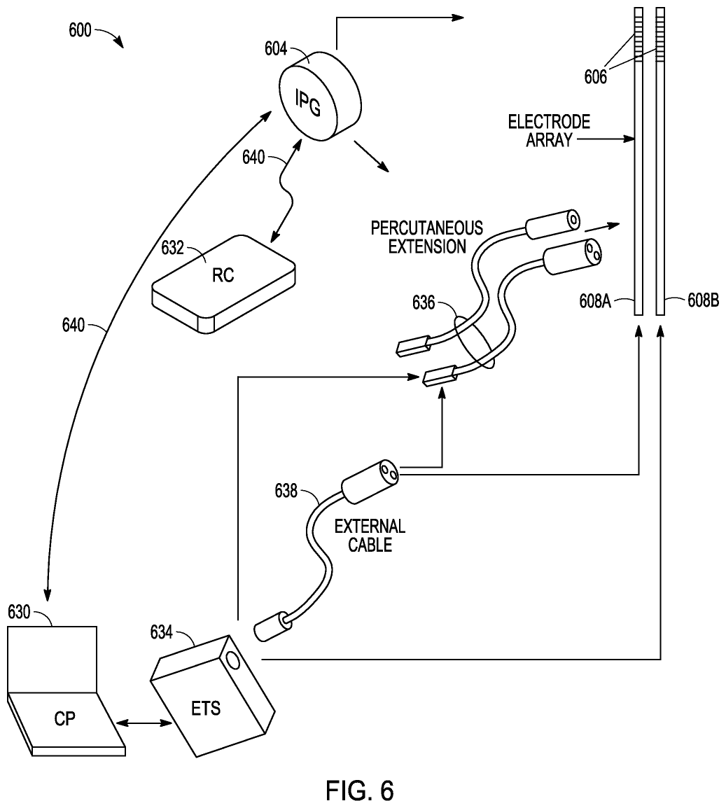

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. IPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes (also referred to as contacts) 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC 632 can be a dedicated device or a general-purpose device configured to perform the functions of RC 632, such as a smartphone, a tablet computer, or other smart/mobile device.

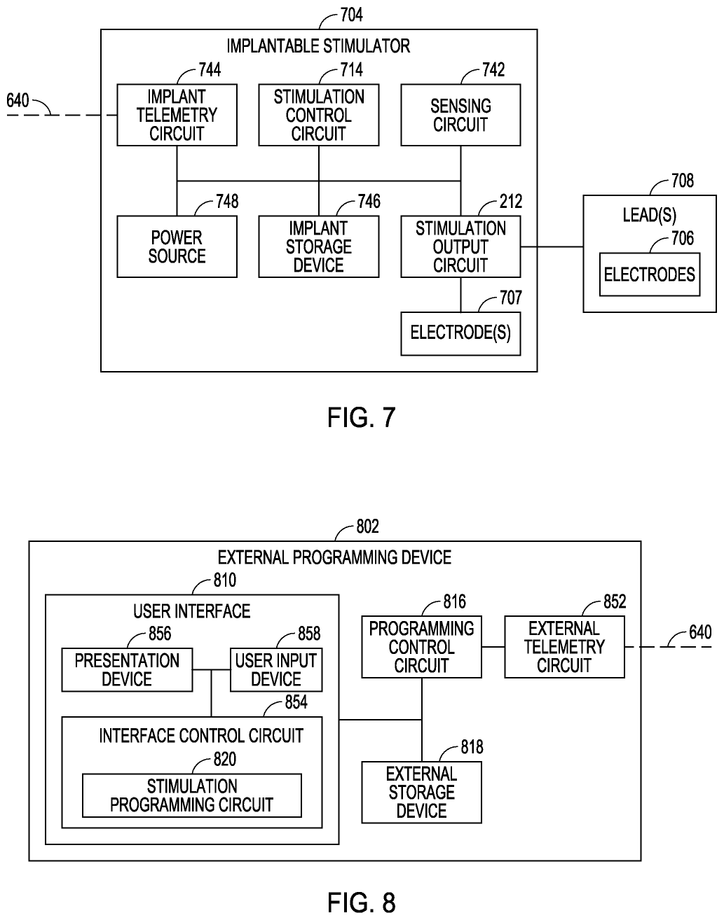

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes (also referred to as contacts) 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more ESG signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time (and allows the user to do so) to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes a stimulation control circuit 820.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

Stimulation control circuit 820 represents an example of stimulation programming circuit 320 and can be configured to provide for closed-loop control of steering of stimulation field according to the present subject matter. In various embodiments, User interface 810 can be configured to allow the user and/or the patient to drive the steering, with stimulation control circuit 820 adjusting the stimulation parameters for substantially maintaining a level of paresthesia while the stimulation field is being moved.

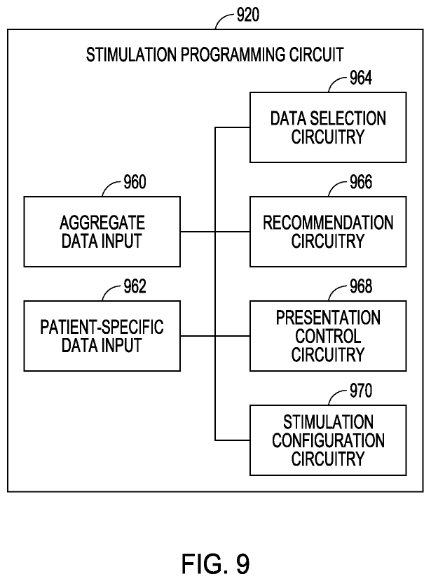

FIG. 9 illustrates an embodiment of a stimulation programming circuit 920 in a programming device of a neurostimulation system. Stimulation programming circuit 920 can represent an example of stimulation programming circuit 320 and can be configured for determining settings of a stimulation device (e.g., stimulation device 104 or 204, IPG 404, 504, or 604, or implantable stimulator 704) for a patient using aggregate data collected from a patient population and patient-specific information collected from the patient. The settings can include a stimulation configuration including stimulation parameters defining one or more stimulation waveforms and one or more stimulation fields. The stimulation device is to be programmed to deliver neurostimulation according to the stimulation configuration. In various embodiments, stimulation programming circuit 920 can be implemented in a programming device such as CP 630, a device (e.g., a computer or mobile device) configured to support the programming of an IPG using CP 630, or both CP 630 and the device.

Stimulation programming circuit 920 can include an aggregate data input 960, a patient-specific data input 962, data selection circuitry 964, recommendation circuitry 966, presentation control circuitry 968, and stimulation configuration circuitry 970. Aggregate data input 960 can receive the aggregate data collected from the patient population. Patient-specific data input 962 can receive the patient-specific data collected from the patient. Data selection circuitry 964 can select relevant data from the received aggregate data according to selection criteria. The selection criteria can be determined based on the collected patient-specific information and/or other information specific to the patient, such as diagnostic information relevant to the patient's indications for neurostimulation and therapeutical goals of neurostimulation for the patient. Recommendation circuitry 968 can generate a target recommendation based on the selected relevant data and the patient-specific data. Presentation control circuitry 968 can determine a data presentation (e.g., to be presented on a user interface such as user interface 810) based on the selected relevant data and the patient-specific data. Stimulation configuration circuitry 970 can determine the stimulation configuration using the target recommendation.

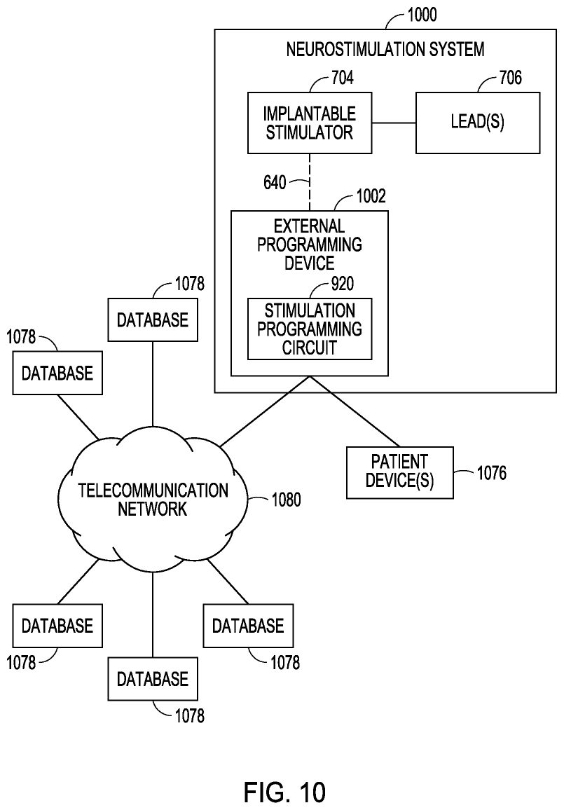

FIG. 10 illustrates an embodiment a neurostimulation system 1000 communicatively coupled to various databases 1078 via a telecommunication system 1080. System 100 can include implantable stimulator 704 coupled to lead(s) 706 and communicatively coupled to an external programming device 1002, which can be implemented in external programming device 802 and includes stimulation programming circuit 920. Stimulation programming circuit 920 can be used in the process of determining the settings of implantable stimulator including the stimulation configuration for the patient and/or the process of positioning lead(s) 706 in the patient. Data bases 1078 can include any number and types of databases containing the aggregate data collected from the patient as well as portions of the patient-specific data (e.g., the patient's medical records). Each database 1078 can belong to a user, a group of users, or an organization participating in the care of patients receiving therapies including neurostimulation. One or more patient devices 1078 can be used to collect the patient-specific information. Examples of patient device(s) 1078 include sensors placed in, on, or near the patient for sensing information from the patient or the patient's environment and portable or mobile devices for the patient to enter information. Telecommunication system 1080 can provide for communications between external programming device 1002 and databases 1078 using one or more wireless communication links or networks such as the Internet, intranets, cellular networks, Bluetooth, and the like. Additionally, when needed, data can also be transferred from each of data bases 1078 to external programming device using portable data storage media. In various embodiments, external programming device 1002 can be implemented as one or more devices, such as being implemented in CP 630, a computer or mobile device, or both CP 630 and the computer or mobile device.

In various embodiments, the aggregate data can include data collected from each patient of the patient population directly (e.g., signals sensed from each patient and/or answers to questions presented to each patient) and/or indirectly (e.g., operational information extracted from each device used to treat the patients and/or information extracted from each patient's medical record). The patient-specific data can include data collected from the patient directly (e.g., signals sensed from the patient and/or answers to questions presented to the patient) and/or indirectly (e.g., operational information extracted from a device used to treat the patient and/or information extracted from the patient's medical record). When external programming device 802 is configured to be external programming device 1002, with stimulation programming circuit 320 configured to include stimulation programming circuit 920, external storage device 818 can store the aggregate data and the patient-specific data, and programming control circuit 816 can generate information for programming implantable stimulator 704 to control the delivery of the neurostimulation according to the stimulation configuration.

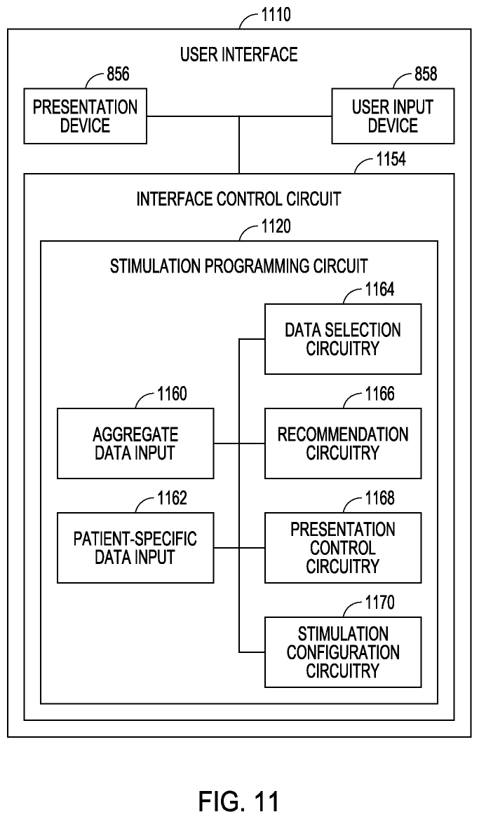

FIG. 11 illustrates an embodiment of a user interface 1110 of an external programming device of an implantable neurostimulation system, such as external programming device 1002. User interface 1110 can represent an example of user interface 810 configured to be the user interface of external programming device 1002 to allow a user to control a processing for programming implantable stimulator 704. User interface 1110 can include presentation device 856, user input device 858, an interface control circuit 1154. Interface control circuit 1154 can control operations of user interface 1110 and includes stimulation programming circuit 1120, which can represent an example of stimulation programming circuit 920. Stimulation programming circuit 1120 can include an aggregate data input 1160, a patient-specific data input 1162, data selection circuitry 1164, recommendation circuitry 1166, presentation control circuitry 1168, and stimulation configuration circuitry 1170.

Aggregate data input 1160 can receive the aggregate data collected from the patient population. The aggregate data can be collected directly and/or indirectly from each patient of the patient population, as discussed above. Patient-specific data input 1162 can receive the patient-specific data collected from the patient. The patient-specific data can be collected directly and/or indirectly from the patient, as discussed above.

Data selection circuitry 1164 can represent an example of data selection circuit 964 and can select relevant data from the received aggregate data according to selection criteria. In various embodiments, the selection criteria can be manually set by the user using user input device 858 and data selection circuitry 1164, automatically determined by data selection circuitry 1164 using the received patient-specific data, or semi-automatically determined by data selection circuitry 1164 using the received patient-specific data and input received from the user using user input device 858. The selection criteria are determined for selecting data useful for various reasons in the process of programming stimulation stimulator for the patient and allow for customization of the stimulation configuration for the patient. In one example, the selection criteria can be manually, automatically, or semi-automatically determined for selecting the relevant data based on profiles including collective patient profiles and a personal profile of the patient. The collective patient profiles are each extracted from the aggregate data for a patient of the patient population, The personal profile is extracted from the patient-specific information. Each profile of the collective patient profiles and the personal profile can include diagnostic and/or therapeutic information. The selection criteria can be determined for selecting the relevant data from patients having similar profiles (e.g., profiles including age, gender, medications prescribed, etiology, family medical history, symptoms, side effects, response to treatment, stimulation target, brain anatomy, physiologic signals, leads implanted, and/or stimulator implanted). In another example, the selection criteria can be manually, automatically, or semi-automatically determined for selecting the relevant data based on the profiles and specified parameter ranges. Examples of specified parameter ranges include a list of selected users, a list of selected clinics, and values ranges of diagnostic parameters (e.g., sensed parameters and data resulting from patient examinations) and/or therapeutic parameters (e.g., parameters of the stimulation configuration and lead placement). In yet another example, the selection criteria can be manually, automatically, or semi-automatically determined for selecting the relevant data based on the profiles, or the profiles and the specified parameter ranges, and further based on the number of qualified patients of the patient population. The qualified patients can include patients whose data are selected based on the profiles or the profiles and the specified parameter ranges. The selection criteria can be determined for selecting the relevant data being data of the qualified patients when the number of the qualified patients reaches a threshold number (e.g., 100 patients). This number can be determined, for example, to ensure that the size of the patient population from which the relevant data are selected is considered statistically significant for the purpose of using the aggregate data in the process of programming implantable stimulator 704.

Recommendation circuitry 1166 can represent an example of recommendation circuitry 966 and can generate a target recommendation based on the selected relevant data and the patient-specific data. The “target recommendation” can include one or more recommendations for settings of the stimulation device to be programmed (e.g., implantable stimulator 704) and/or one or more recommendations for lead placement (e.g., placement of lead(s) 708 for use with implantable stimulator 704) in the patient to achieve a therapeutic target (e.g., a goal or intended outcome of the neurostimulation). The selection criteria can be determined using data selection circuitry based on information required for recommendation circuitry to generate the target recommendation. In various embodiments, the target recommendation can include an input recommendation or an output recommendation. For example, the output recommendation can include recommended values of stimulation parameters of the stimulation configuration, and the input recommendation can include data required by an intermediate step in generating the output recommendation from the selected relevant data and the patient-specific data. In one embodiment, recommendation circuitry 1166 generates the input recommendation using the selected relevant data and the patient-specific data, to be further processed for generating the output recommendation. In another embodiment, recommendation circuitry 1166 generates the output recommendation directly through an automatic process using the selected relevant data and the patient-specific data.

Presentation control circuitry 1168 can represent an example of presentation control circuitry 968 and can determine a data presentation using presentation device 856 based on the selected relevant data and the patient-specific data. In various embodiments, the data presentation can include the target recommendation including the input recommendation (e.g., populating input fields of a computerized tool for generating the output recommendation) or the output recommendation (e.g., parameters of the stimulation configuration), depending on the output of recommendation circuitry 1166. In one example, the data presentation includes a visual representation of portions of the selected relevant data and/or data derived from the selected relevant data (e.g., ranges, medians, and/or averages). In another example, the data presentation includes visual representation showing overlapping between the target recommendation and a corresponding current state of the system to allow for increase of the overlapping. The “current state of the system” can include current settings of the stimulation device (e.g., implantable stimulator 704) as programmed for the patient and/or current lead placement (e.g., lead(s) 708 connected to implantable stimulator 704) in the patient. In yet another example, the data presentation includes a measure of likelihood (e.g., a probability) determined based on comparison of the target recommendation to the corresponding current state of the system.

Stimulation configuration circuitry 1170 can represent an example of stimulation configuration circuitry 970 and can determine the stimulation configuration using the target recommendation. In one embodiment, in which the target recommendation includes the input recommendation, stimulation configuration circuitry 1170 can receive the input recommendation and execute an algorithm determining the output recommendation based on the input recommendation. For example, a computerized tool can be installed in the external programming device for determining the output recommendation (e.g., settings of the implantable stimulator) based on an initial setup that can include default and/or recommended values for the stimulation parameters. Stimulation configuration circuitry 1170 can execute the algorithm underlying the computerized tool by using the input recommendation as the initial setup or a recommendation for the initial setup. The present subject matter can be compatible with such computerized tools that are existing and/or being separately developed. The selection criteria can be determined using data selection circuitry 1164 based on information required for generating the input setup for the computerized tool. The input recommendation facilitates the use of the computerized tool and improves the quality of the stimulation configuration determined by the computerized tool. After the output recommendation is determined, presentation control circuitry 1168 can cause the output recommendation to be presented to the user using presentation device 856. Stimulation configuration circuitry 1170 can then receive a user command entered using user input device 858 and determine the stimulation configuration for programming implantable stimulator 704 based on the output recommendation and the user command. In another embodiment, in which the target recommendation includes the output recommendation, presentation control circuitry 1168 can cause the output recommendation to be presented to the user using presentation device 856. Stimulation configuration circuitry 1170 can then receive a user command entered using user input device 858 and determine the stimulation configuration for programming implantable stimulator 704 based on the output recommendation and the user command. In both embodiments, the user command can direct stimulation configuration circuitry 1170 to accept the output recommendation, accept the output recommendation with modifications, or reject the output recommendation.

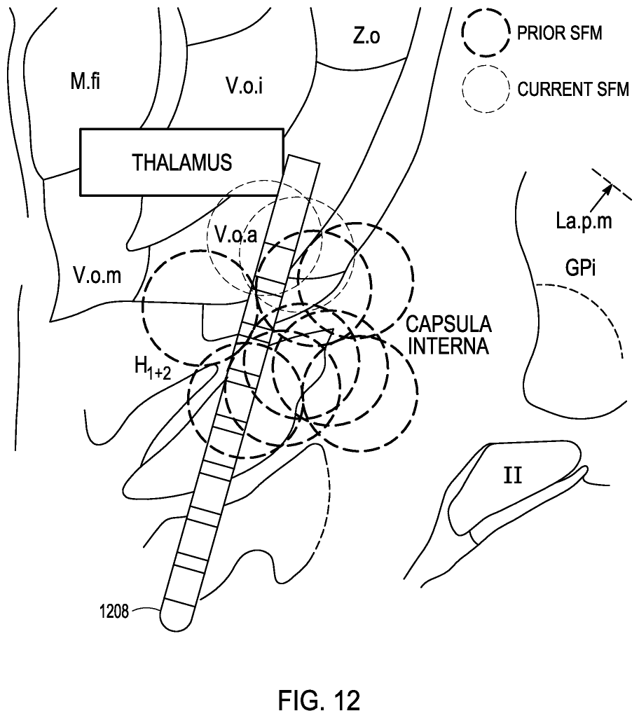

Some examples are discussed, with references to FIGS. 12-14, to illustrate the selection of the relevant data and the data presentation using stimulation programming circuit 1120 in some applications. These examples are discussed for the purpose of illustration only (i.e., not for restriction). FIGS. 12-14 each illustrates a portion of the data presentation as displayed on a screen such as the screen of presentation device 856.

FIG. 12 illustrates an embodiment of portions of a display for guiding lead placement, such as on the screen of presentation device 856, during implantation of an implantable stimulator, such as implantable stimulator 704. Stimulation field models (SFMs), also referred to as volumes of tissue activated (VTAs) or volumes of activation (VOAs), each represent a volume of the tissue activated by the delivery of the neurostimulation depending on the lead placement (i.e., location of electrodes) and can be used to guide the lead placement. An example of creating an SFM based on stimulation parameters is discussed in U.S. Pat. No. 8,849,411, “USER-DEFINED GRAPHICAL SHAPES USED AS A VISUALIZATION AID FOR STIMULATOR PROGRAMMING”, assigned to Boston Scientific Neuromodulation Corporation, which is herein incorporated by reference in its entirety.

In the illustrated embodiment, the selected relevant data and the data presentation include “prior SFMs” which are SFMs associated with lead positions collected from patients having implanted with implantable stimulators and leads. The data presentation also includes “current SFMs” which are SFMs associated with the lead being placed in the patient (e.g., as part of the current state of the system), determined using the patient-specific data. FIG. 12 shows the prior SFMs and the current SFMs being displayed simultaneously with a lead 1208 (e.g., a lead of lead(s) 708) showing its position associated with the current SFMs. The selection criteria can be determined for the selected relevant data and the data presentation data to include only prior SFMs associated with a specified range of therapeutic effectiveness (e.g., exceeding a threshold measure of intended improvement of the patients' symptoms). The data presentation can include highlighting of overlapping volume(s) of the prior SFMs. The data presentation can include a probability or other statistical measure to show a relative level of confidence in a recommendation determined using the selected relevant data. For example, the data presentation can show a certain percentage (e.g., 20%) of the patients having SFMs overlapping in a region suggest moving the lead to a direction resulting in the current SFMs covering that region. The target recommendation includes such overlapping volume(s) to recommend where the lead should be placed or how a lead placement should be adjusted during the implantation of implantable stimulators and leads.

In a similar example, the aggregate data include pain areas (anatomic areas). The selected relevant data and the data presentation include such pain areas to be displayed with pain areas estimated using the patient-specific data of the patient for recommending lead placement for the patient, for example on the patient's spinal cord for delivering SCS.

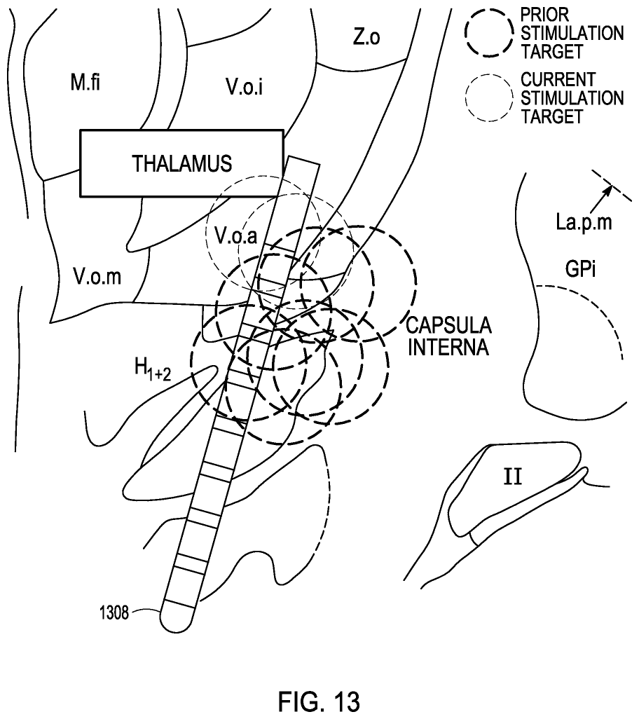

FIG. 13 illustrates an embodiment of portions of a display for identifying stimulation targets, such as on the screen of presentation device 856. The stimulation targets are each an area or a volume of tissue to which the neurostimulation (e.g., electrical pulses) is to be delivered. The selected relevant data and the data presentation include “prior stimulation targets” which are target volumes associated with lead positions collected from the patients having implanted with implantable stimulators and leads. The data presentation can also include “current stimulation targets” associated with a current stimulation configuration set for the patient (e.g., part of the current state of the system). FIG. 13 shows the prior stimulation targets and the current stimulation targets being displayed simultaneously with a lead 1308 (e.g., a lead of lead(s) 708) showing its position associated with the current stimulation targets. This allows the user to import prior stimulation targets from the aggregated data to be used to determine the input recommendation, which includes one or more stimulation targets derived from the prior stimulation targets. A computerized tool can generate the output recommendation (e.g., a recommended stimulation configuration) based on the prior stimulation targets. This allows the aggregate data to be used for optimizing the stimulation targets for therapeutic effectiveness while avoiding side effects. If the user has created a stimulation target and set a specific weighting factor for that stimulation target, the aggregate data can be used to recommend adjustments to the created stimulation target or to the weighting factor to increase overlap between the created stimulation target and stimulation targets recommended based on the aggregate data.

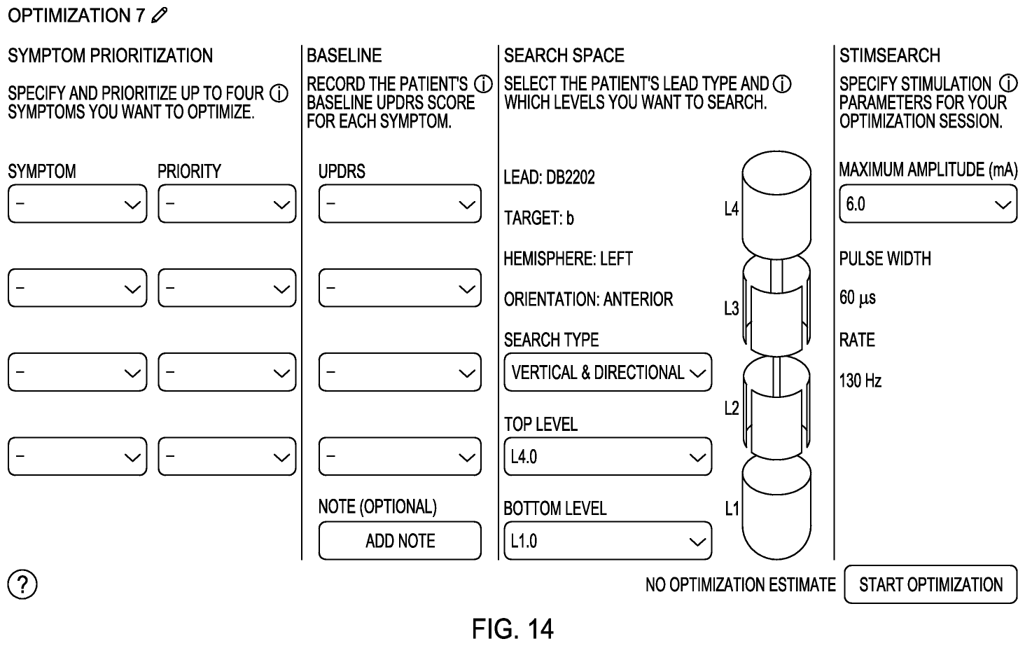

FIG. 14 illustrates an embodiment of portions of a display for recommending setup features on a user interface, such as on the screen of presentation device 856. The setup features are the input recommendation that can be used by a computerized tool for generating the output recommendation. FIG. 14 shows a setup screen of a computerized tool that receives setup features (the input recommendation) and generates recommendations for settings of an implantable stimulator (the output recommendation) based on the setup features. As illustrated in FIG. 14, such a setup screen can be complicated, with many setup features fields to be filled. While information collected using the setup screen is important for generating the recommendations and provides immense flexibility for different setups, it may appear daunting to the user. The selection criteria can be determined based on the information needed to fill the fields in the setup screen. The data presentation includes data extracted and/or derived from the selected relevant data and the patient-specific data to fill the setup screen.

In one example, as shown in FIG. 14, the setup features include symptoms each associated with a weight (“priority”). Symptoms to enter can include the most severe symptoms that the patient has as well as the likelihood that those symptoms would respond to treatment. While the symptom selection can be entered by the user, patient input (e.g., received using a patient application installed on a smartphone) can be used to automatically populate these fields with the top four symptoms. The weights can be derived from a combination of the patient-specific data and the selected relevant data associated with patients having similar symptoms. Such data can be used to predict therapeutical outcomes and to set the weights based on the predicted therapeutical outcomes. If the user chooses a symptom that appears unlikely to respond to the treatment, the user can receive a notification to consider selecting another symptom or lowering the weight for the chosen symptom.

In another example, also as shown in FIG. 14, the setup features include search space (for lead placement) and stimulation parameters search (“StimSearch”, for setting values of stimulation parameters such as pulse amplitude, pulse width, and pulse rate). The setup features can be used to limit each of search space and stimulation parameters search to a range. A smaller range can make the search more efficient. The setup features provide starting ranges used in a search process for finding an optimal setting for each of the lead placement and stimulation parameters. Such starting ranges can be reduced or minimized using an input recommendation determined using the selected relevant data and the patient-specific data to make the search process more efficient. The input recommendation can be used to fill the search space and stimulation parameters search fields automatically or to adjust user-determined or default values and selections in these fields.

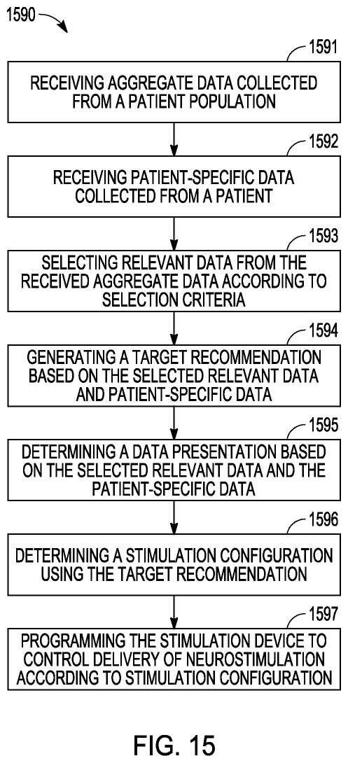

FIG. 15 illustrates an embodiment of a method 1590 for determining settings of a stimulation device for a patient using aggregate data collected from a patient population and patient-specific information collected from the patient. In one embodiment, method 1590 is performed using external programming device 1002 in system 1000. In one embodiment, a non-transitory computer-readable storage medium includes instructions, which when executed by a machine (e.g., external programming device 1002), cause the machine to perform method 1590. Examples of the stimulation device include stimulation device 104 or 204, IPG 404, 504, or 604, or implantable stimulator 704.

At 1591, aggregate data are collected from a patient population. At 1592, patient-specific data are collected from the patient. The aggregate data can be collected directly and/or indirectly from each patient of the patient population, as discussed above. The patient-specific data can be collected directly and/or indirectly from the patient, as discussed above.

At 1593, relevant data are selected (e.g., using a processor of external programming device 1002) from the received aggregate data according to selection criteria. The selection criteria can be determined for selecting the relevant data based on profiles including collective patient profiles and a personal profile of the patient. The collective patient profiles are each extracted from the aggregate data for a patient of the patient population. The personal profile is extracted from the patient-specific information. Each profile of the collective patient profiles and the personal profile can include diagnostic information and/or therapeutic information. The selection criteria can also be determined for selecting the relevant data further based on specified parameter ranges and/or a number of qualified patients of the patient population.

At 1594, a target recommendation is generated (e.g., using a processor of external programming device 1002) based on the selected relevant data and the patient-specific data. In various embodiments, the target recommendation can include an input recommendation or an output recommendation. The output recommendation can include recommended values of stimulation parameters of the stimulation configuration, and the input recommendation can include data required by an intermediate step in generating the output recommendation from the selected relevant data and the patient-specific data. An example of the input recommendation is recommended input to a computerized tool for automatically generating the output recommendation.

At 1595, a data presentation is determined (e.g., using a processor of external programming device 1002) based on the selected relevant data and the patient-specific data, to be presented using a user interface (e.g., user interface 1110). In various embodiments, the data presentation can include, for example, a visual representation of at least one of portions of the selected relevant data or data derived from the selected relevant data using the presentation device, a visual representation showing overlapping between the target recommendation and corresponding current state of the system, and/or a visual representation including a measure of likelihood of an intended outcome of the delivery of the neurostimulation determined based on a comparison of the target recommendation to the corresponding state of the system.

At 1596, a stimulation configuration is determined (e.g., using a processor of external programming device 1002) using the target recommendation. The stimulation configuration includes stimulation parameters defining one or more stimulation waveforms and one or more stimulation fields. In various embodiments, this stimulation configuration is presented as a recommended stimulation configuration to the user. The user can finalize a stimulation configuration for programming the stimulation device by accepting the recommended stimulation configuration with no modification, accepting the recommended stimulation configuration with modifications, or rejecting the recommended stimulation configuration and start another process of determining the stimulation configuration.

At 1597, the stimulation device is programmed to control the delivery of the neurostimulation according to the stimulation configuration. In various embodiments, method 1590 can be repeated, for example periodically and/or as need, for adjusting the stimulation configuration to maintain or improve effectiveness of the neurostimulation.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to a patient using a stimulation device configured to deliver the neurostimulation, the system comprising:
a programming control circuit configured to generate information for programming the stimulation device to control the delivery of the neurostimulation according to a stimulation configuration including stimulation parameters defining one or more stimulation waveforms and one or more stimulation fields; and
a user interface including a presentation device, a user input device, and a stimulation programming circuit coupled to the presentation device and the user input device and configured to receive aggregate data collected from a patient population, to receive patient-specific data collected from the patient, and to determine the stimulation configuration, the stimulation programming circuit including:
data selection circuitry configured to select relevant data from the received aggregate data according to selection criteria;
recommendation circuitry configured to generate a target recommendation based on the selected relevant data and the patient-specific data;
presentation control circuitry configured to determine a data presentation using the presentation device based on the selected relevant data and the patient-specific data; and
stimulation configuration circuitry configured to determine the stimulation configuration using the target recommendation.

2. The system of claim 1, wherein the data selection circuitry is further configured to determine the selection criteria automatically using the received patient-specific data and information required for generating the target recommendation.

3. The system of claim 2, wherein the data selection circuitry is configured to determine the selection criteria for selecting the relevant data based on profiles including collective patient profiles and a personal profile of the patient, the collective patient profiles each extracted from the aggregate data for a patient of the patient population, the personal profile extracted from the patient-specific information, each profile of the collective patient profiles and the personal profile including at least one of diagnostic information or therapeutic information.

4. The system of claim 1, wherein the recommendation circuitry is configured to generate an input recommendation of the target recommendation, and the stimulation configuration circuitry is configured to execute an algorithm configured to determine an output recommendation automatically based on the input recommendation, to receive a user command, and to determine the stimulation configuration using the output recommendation and the user command.

5. The system of claim 1, wherein the presentation circuitry is configured to present visual representation of at least one of portions of the selected relevant data or data derived from the selected relevant data using the presentation device.

6. The system of claim 1, wherein the presentation circuitry is configured to present visual representation showing overlapping between the target recommendation and corresponding current state of the system.

7. The system of claim 1, wherein the presentation circuitry is configured to present a measure of likelihood of an intended outcome of the delivery of the neurostimulation determined based on a comparison of the target recommendation to the corresponding current state of the system.

8. The system of claim 1, wherein the presentation control circuit is configured to determine the data presentation using portions of the selected relevant data for guiding lead placement during implantation of the stimulation device, the data presentation including stimulation field models associated with lead positions collected from patients of the patient population having implanted stimulation devices, the stimulation field models (SFMs) each representing a volume of tissue activated by the delivery of the neurostimulation.

9. The system of claim 1, wherein the presentation control circuit is configured to determine the data presentation using portions of the selected relevant data for guiding an identification of a stimulation target being an area or a volume of tissue to which the neurostimulation is to be delivered, the data presentation including prior stimulation targets associated with lead positions collected from patients of the patient population having implanted stimulation devices.

10. The system of claim 1, wherein the presentation control circuit is configured to determine the data presentation using portions of the selected relevant data, the data presentation including setup features determined using the portions of the selected relevant data and used as input used for generating the target recommendation automatically.

11. A method for delivering neurostimulation to a patient using a stimulation device configured to deliver the neurostimulation, the method comprising:
receiving aggregate data collected from a patient population;
receiving patient-specific data collected from the patient;
selecting relevant data from the received aggregate data according to selection criteria using a processor;
generating a target recommendation based on the selected relevant data and the patient-specific data using the processor;
determining a data presentation based on the selected relevant data and the patient-specific data using the processor, to be presented using a user interface;
determining a stimulation configuration using the target recommendation using the processor, the stimulation configuration including stimulation parameters defining one or more stimulation waveforms and one or more stimulation fields; and
programming the stimulation device to control the delivery of the neurostimulation according to the stimulation configuration.

12. The method of claim 11, wherein determining the data presentation comprises determining a visual representation of at least one of portions of the selected relevant data or data derived from the selected relevant data using the presentation device.

13. The method of claim 11, wherein determining the data presentation comprises determining a visual representation showing overlapping between the target recommendation and corresponding current state of the system.

14. The method of claim 11, wherein determining the data presentation comprises determining a visual representation including a measure of likelihood of an intended outcome of the delivery of the neurostimulation determined based on a comparison of the target recommendation to the corresponding current state of the system.

15. The method of claim 11, further comprising determining the selection criteria for selecting the relevant data based on profiles including collective patient profiles and a personal profile of the patient, the collective patient profiles each extracted from the aggregate data for a patient of the patient population, the personal profile extracted from the patient-specific information, each profile of the collective patient profiles and the personal profile including at least one of diagnostic information or therapeutic information.

16. The method of claim 15, wherein determining the selection criteria comprises determining the selection criteria for selecting the relevant data further based on at least one of specified parameter ranges or a number of qualified patients of the patient population.

17. The method of claim 11, wherein determining the data presentation comprises determining the data presentation using portions of the selected relevant data for guiding lead placement during implantation of the stimulation device, the data presentation including stimulation field models associated with lead positions collected from patients of the patient population having implanted stimulation devices, the stimulation field models (SFMs) each representing a volume of tissue activated by the delivery of the neurostimulation.

18. The method of claim 11, wherein determining the data presentation comprises determining the data presentation using portions of the selected relevant data for guiding an identification of a stimulation target being an area or a volume of tissue to which the neurostimulation is to be delivered, the data presentation including prior stimulation targets associated with lead positions collected from patients of the patient population having implanted stimulation devices.

19. The method of claim 11, wherein determining the data presentation comprises determining the data presentation using portions of the selected relevant data, the data presentation including setup features determined using the portions of the selected relevant data and used as input used for generating the target recommendation automatically.

20. A non-transitory computer-readable storage medium including instructions, which when executed by a system, cause the system to perform a method for delivering neurostimulation to a patient using a stimulation device configured to deliver the neurostimulation, the method comprising:
receiving aggregate data collected from a patient population;
receiving patient-specific data collected from the patient;
selecting relevant data from the received aggregate data according to selection criteria;
generating a target recommendation based on the selected relevant data and the patient-specific data;
determining a data presentation based on the selected relevant data and the patient-specific data using the processor, to be presented using a user interface;
determining a stimulation configuration using the target recommendation, the stimulation configuration including stimulation parameters defining one or more stimulation waveforms and one or more stimulation fields; and
generating information for programming the stimulation device to control the delivery of the neurostimulation according to the stimulation configuration.
